(19) United States
(12) Patent Application Publication
(10) Pub. No.: US 2006/0051334 A1
(43) Pub. Date: Mar. 9, 2006

(54) Injection of bone marrow-derived conditioned medium for angiogenesis

(75) Inventors: Ran Kornowski, Ramat-Hasharon (IL); Shmuel Fuchs, Rockville, MD (US); Stephen Epstein, Rockville, MD (US); Martin B. Leon, New York, NY (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: MYOCARDIAL THERAPEUTICS, INC., La Jolla, CA (US)

(21) Appl. No.: 11/117,607

(22) Filed: Apr. 27, 2005

Related U.S. Application Data
(63) Continuation-in-part of application No. 10/160,514, filed on May 30, 2002, now abandoned.
Application No. 10/160,514 is a continuation-in-part of application No. 09/868,411, filed on Jun. 14, 2001.
(60) Provisional application No. 60/566,332, filed on Apr. 28, 2004.
(60) Provisional application No. 60/138,379, filed on Jun. 9, 1999.
(60) Provisional application No. 60/126,800, filed on Mar. 30, 1999.

(51) Int. Cl. A61K 48/00

(52) U.S. Cl. 424/93.21; 514/44

(57) ABSTRACT

Methods are provided for promoting formation of collateral blood supply at an ischemic site in tissue by culturing early attaching cells derived from growth of bone marrow aspirate in vitro, collecting early attaching cells produced by the bone marrow culture and injecting conditioned medium produced by culture of the early attaching cells into an ischemic site in heart or limb. The preferred early attaching cells for use in the invention methods are marrow-derived stromal cells. Any donor's bone marrow can be used in preparation of the conditioned medium. Optionally, the early attaching cells can be transfected with an angiogenesis promoting transgene encoding hypoxia inducing factor 1 alpha, a fibroblast growth factor and/or a nitric oxide synthase. Conditioned media containing angiogenic cytokines produced such cells are also provided for injection into tissue, such as heart or peripheral limb muscle, requiring formation of collateral blood supply.

### RELATED APPLICATIONS

This application relies for priority on provisional application 60/566,332, filed Apr. 28, 2004, and is related to Continuation-in-Part Application of U.S. patent application Ser. No. 10/160,514, filed Jun. 6, 2002, which is a Continuation-in-Part Application of U.S. patent application Ser. No. 09/868,411, filed Jun. 14, 2001, which was the National Stage of International Application No. PCT/US00/08353, filed Mar. 30, 2000, which relies for priority upon U.S. Provisional Patent Application Ser. Nos. 60/138,379, filed Jun. 9, 1999, and 60/126,800, filed Mar. 30, 1999, now abandoned, each of which is incorporated herein in its entirety.

### FIELD OF THE INVENTION

This application is directed to methods for treating a site of damaged blood supply by injecting bone marrow and bone marrow cell products. More specifically, this invention is directed to intramyocardial injection of bone marrow derived conditioned medium produced by culturing bone marrow cells, to enhance collateral blood vessel formation (angiogenesis) and tissue perfusion.

### BACKGROUND OF THE INVENTION

The use of recombinant genes or growth factors to enhance myocardial collateral blood vessel function may represent a new approach to the treatment of cardiovascular disease. Kornowski, R., et al., “Delivery strategies for therapeutic myocardial angiogenesis,” Circulation 2000; 101:454-458. Proof of concept has been demonstrated in animal models of myocardial ischemia, and clinical trials are underway. Unger, E. F., et al., “Basic fibroblast growth factor enhances myocardial collateral flow in a canine model,” Am J Physiol 1994; 266:H1588-1595; Banai, S. et al., “Angiogenic-induced enhancement of collateral blood flow to ischemic myocardium by vascular endothelial growth factor in dogs,” Circulation 1994; 83-2189; Lazarous, D. F., et al., “Effect of chronic systemic administration of basic fibroblast growth factor on collateral development in the canine heart,” Circulation 1995; 91:145-153; Lazarous, D. F., et al., “Comparative effects of basic development and the arterial response to injury,” Circulation 1996; 94:1074-1082; Giordano, F. J., et al., “Intracoronary gene transfer of fibroblast growth factor-5 increases blood flow and contractile function in an ischemic region of the heart,” Nature Med 1996; 2:534-9. Guzman, R. J., et al., “Efficient gene transfer into myocardium by direct injection of adenovirus vectors,” Circ Res 1993; 73:1202-7; Mack, C. A., et al., “Biologic bypass with the use of adenovirus-mediated gene transfer of the complementary deoxyribonucleic acid for VEGF-121, improves myocardial perfusion and function in the ischemic porcine heart,” J Thorac Cardiovasc Surg 1998; 115:168-77.

The effect of direct intra-operative intramyocardial injection of angiogenic factors on collateral function has been studied in animal models of myocardial ischemia. Open chest, transepicardial administration of an adenoviral vector containing a transgene encoding an angiogenic peptide resulted in enhanced collateral function. (Mack et al., supra.) Angiogenesis was also reported to occur with direct intramyocardial injection of an angiogenic peptide or a plasmid vector during open-heart surgery in patients. Schumacher, B., et al., “Induction of neoangiogenesis in ischemic myocardium by human growth factors. First clinical results of a new treatment of coronary heart disease,” Circulation 1998; 97:645-650; Losordo, D. W., et al., “Gene therapy for myocardial angiogenesis: initial clinical results with direct myocardial injection of phVEGF165 as sole therapy for myocardial ischemia,” Circulation 1998; 98:2800.

Despite the promising hope for therapeutic angiogenesis as a new modality to treat patients with coronary artery disease, there is still a huge gap regarding what specific strategy will optimally promote a clinically relevant therapeutic angiogenic response. Moreover, it is unclear which one (or more) out of multiple angiogenic growth factors may be associated with a beneficial angiogenic response. In addition, the use of different tissue delivery platforms, e.g., proteins, adenovirus, or “naked” DNA, to promote the optimal angiogenic response has remained an open issue.

Therefore, there is a need in the art for more and better compositions and methods for enhancing angiogenesis in tissue, such as muscle tissue, in which development of collateral arteries for tissue perfusion is desired.

### SUMMARY OF THE INVENTION

The present invention is based on the premise that multiple complex processes, involving the differential expression of dozens if not hundreds of genes, are necessary for optimal collateral development. Based on this concept, it follows that optimal development of collateral blood vessels and tissue perfusion cannot be achieved by the administration of single genes whose encoded products are known to be related to angiogenesis nor, because of the complexity of the angiogenesis processes, by the administration of a combination of angiogenesis-related genes. This invention relies on the capacity of bone marrow cells to secrete the growth factors and cytokines involved in angiogenesis in a time and concentration-dependent coordinated and appropriate sequence.

Most currently tested therapeutic approaches have focused on a single angiogenic growth factor (e.g., VEGF, FGF, angiopoietin-1) delivered to the ischemic tissue. -This can be accomplished either by delivery of the end product (e.g., protein) or by gene transfer, using diverse vectors. However, it is believed that complex interactions among several growth factor systems are probably necessary for the initiation and maintenance of new blood vessel formation. More specifically, it is believed important to induce a specific localized angiogenic milieu with various angiogenic cytokines interacting in concert and in a time-appropriate manner to initiate and maintain the formation and function of new blood vessels.

Accordingly, in one embodiment, the invention provides methods for promoting collateral blood vessel formation and tissue perfusion in tissue by injecting conditioned medium of bone marrow early attaching cells into the tissue to promote formation of collateral blood vessels in the tissue.

In another embodiment, the invention provides methods for enhancing collateral blood vessel formation in a patient in need thereof by growing bone marrow under suitable culture conditions for a period of time sufficient to promote production by the bone marrow of early attaching cells; transfecting at least a portion of the early attaching cells with a vector comprising a polynucleotide that encodes one or more agents selected from angiogenic cytokines, growth factors and mammalian angiogenesis-promoting factors for expression by the early attaching cells, and culturing the transfected early attaching cells in a culture medium and for a time suitable to allow expression by the cells of the one or more agents, thereby producing conditioned medium. An effective amount of the transfected early attaching cells and/or the conditioned medium is then directly administered to a desired site in the patient, thereby enhancing collateral blood vessel formation at the site in the patient.

In still another embodiment, the invention provides a therapeutic composition comprising early attaching cells derived from bone marrow, which cells have been transfected with a vector comprising a polynucleotide that encodes one or more agents selected from angiogenic cytokines, growth factors, and angiogenesis-promoting factors. The therapeutic composition can further comprise a conditioned medium in which the transfected cells have been grown in culture for a time sufficient to allow expression of one or more of the transgenic agents as well as other agents normally produced by such cells in culture.

### BRIEF DESCRIPTION OF THE DRAWINGS

FIG. 1 is a graph of the proliferation of PAEC's vs. the quantities of conditioned medium;

FIG. 2 is a graph of the proliferation of endothelial cells vs. the quantities of conditioned medium;

FIG. 3 is a graph of the concentration of VEGF in conditioned medium over a four-week period of time; and

FIG. 4 is a graph of the concentration of MCP-1 in conditioned medium over a four-week period of time.

FIG. 5A is a graph showing in-vitro production of VEGF, MCP-1 and bFGF by CD34+ cells and bone marrow-derived stromal cells from mice.

FIG. 5B is a graph showing % increase of human umbilical vein endothelial cells caused by MSC conditioned media as a function of conditioned media (CM) concentration. -●-=VEGF; -♦-=conditioned medium alone; -o-conditioned medium plus VEGF blocking antibody.

FIG. 6 is a graph showing the effect of bone marrow-derived stromal cells on development of collateral flow when injected into adductor muscles of ischemic hind limb of mice as determined by Laser/Doppler perfusion imaging. Flow is expressed as the ratio of flow in the ischemic limb to flow in the normal hind limb. MSC=marrow-derived stromal cells; Media=non-conditioned media; MAEC=mouse aortic endothelial cells.

FIG. 7 is a graph showing the effect on release of VEGF and bFGF in vitro from mouse marrow-derived stromal cells (MSCs) transfected with an adenovirus encoding HIF-1I-VP 16. (MSC=MSCs alone; hypoxia=hypoxia conditions alone; HIP=MSCs transfected with DNA encoding fusion protein HIF-1α-VP 16. Data represent analysis of at least 3 different MSC populations.

FIG. 8 is a graph showing in vivo flow recovery in mice receiving 1×10⁵ HIF-1α/VP16 transduced MSCs injected into an ischemic hind limb compared to 1×10⁵ non-transduced MSCs (comparison of trends p=0.05 by ANOVA). Cells injected on day 1.

FIG. 9 is a graph showing VEGF values for human coronary artery patients (±SD) under baseline conditions, following hypoxia exposure, and following Ad.HIF-1α/VP16 transduction. * p<0.05 vs normoxia; † p<0.001 vs normoxia; ‡ p<0.001 vs hypoxia. Data of the patient with lowest VEGF expression at baseline are depicted separately.

### DETAILED DESCRIPTION OF THE INVENTION

The bone marrow (BM) is a natural source of a broad spectrum of cytokines (e.g., growth factors) and cells that are involved in the control of angiogenic processes. It is therefore believed that the delivery of autologous bone marrow(ABM) or bone marrow cells derived therefrom, or media derived from these cells while the cells are grown in culture, by taking advantage of the natural ability of these cells to secrete many angiogenic factors in a time-appropriate manner, provides an optimal intervention for achieving therapeutic collateral development in ischemic myocardium.

According to various embodiments of the invention, autologous bone marrow, or cells derived therefrom, or media derived from these cells while the cells are grown in culture, is injected, either as a “stand alone” therapeutic agent or combined with any pharmacologic drug, protein or gene or any other compound or intervention that may enhance bone marrow production of angiogenic growth factors and/or promote endothelial cell proliferation, migration, and blood vessel tube formation. The “combined” angiogenic agents can be administered directly into the patient or target tissue, or incubated ex-vivo with bone marrow prior to injection of bone marrow or bone marrow cells into the patient. As used herein, the term “bone marrow cells” means any cells that are produced by culturing of aspirated bone marrow under cell growth conditions.

Non-limiting examples of these “combined” angiogenic agents are Granulocyte-Monocyte Colony Stimulatory Factor (GM-CSF), Monocyte Chemoattractant Protein 1 (MCP-1), and Hypoxia Inducible Factor-1 (HIF-1).

Bone marrow is also a natural source of a broad spectrum of cytokines, growth factors and angiogenesis-promoting factors that are involved in the control of angiogenic and inflammatory processes. The angiogenic cytokines, growth factors and angiogenesis-promoting factors expressed comprise mediators known to be involved in the maintenance of early and late hematopoiesis (IL-1 alpha and IL-1 beta, IL-6, IL-7, IL-8, IL-11 and IL-13; colony-stimulating factors, thrombopoietin, erythropoietin, stem cell factor, fit 3-ligand, hepatocyte cell growth factor, tumor necrosis factor alpha, leukemia inhibitory factor, transforming growth factors beta 1 and beta 3; and macrophage inflammatory protein 1 alpha), angiogenic factors (fibroblast growth factors 1 and 2, vascular endothelial growth factor) and mediators whose usual target (and source) is the connective tissue-forming cells (platelet-derived growth factor A, epidermal growth factor, transforming growth factors alpha and beta 2, oncostatin M and insulin-like growth factor-1), or neuronal cells (nerve growth factor). Sensebe, L., et al., Stem Cells 1997; 15:133-43. Moreover, it has been shown that VEGF polypeptides are present in platelets and megacaryocytes, and are released from activated platelets together with the release of beta-thromboglobulin. Wartiovaara, U., et al., Thromb Haemost 1998; 80:171-5; Mohle, R., Proc Natl Acad Sci USA 1997; 94:663-8.

There are also indicators to support the concept that angiogenesis is needed to support bone marrow function and development of hematopoietic cells, including stem cells and progenitor cells, that may enter the circulation and target to sites of wound healing and/or ischemia, ultimately contributing to new blood vessel formation. Monoclonal antibodies that specifically recognize undifferentiated mesenchymal progenitor cells isolated from adult human bone marrow have been shown to recognize cell surface markers of developing microvasculature, and evidence suggests such cells may play a role in embryonal angiogenesis. Fleming, J. E., Jr., Dev Dyn 1998; 212:119-32.

Bone marrow angiogenesis may become exaggerated in pathologic states where the bone marrow is being activated by malignant cells (such as in multiple myeloma) where bone marrow angiogenesis has been shown to increase simultaneously with progression of human multiple myeloma cells. Ribatti, D., et al., Br J Cancer 1999; 79:451-5. Moreover, vascular endothelial growth factor (VEGF) has been shown to play a role in the growth of hematopoietic neoplasms such as multiple myeloma, through either a paracrine or an autocrine mechanism. Bellamy, W. T., Cancer Res 1999; 59:728-33; Fiedler, W., Blood 1997; 89:1870-5). It is believed that autologous bone marrow, with its unique native humoral and cellular properties, is a potential source of various angiogenic compounds. This natural source of “mixed” angiogenic cytokines can surprisingly be utilized as a mixture of potent interactive growth factors to produce therapeutic angiogenesis and/or myogenesis; use of the cells per se could provide a more sustained source of these natural angiogenic agents.

In addition, it has now been surprisingly discovered that the media in which such bone marrow cells are cultured contains such a mixture of interactive growth factor proteins that produce therapeutic angiogenesis and/or myogenesis. Moreover, the therapeutic effects can be produced by culturing non-autologous bone marrow cells for a time suitable to allow production by the bone marrow cells of the interactive growth factor proteins and delivering the “conditioned” media to a region of ischemic tissue to produce the therapeutic angiogenesis and/or myogenesis. In fact, it is an “unexpected result” that when transfected early attaching bone marrow cells, such bone marrow cells and conditioned medium obtained by growing the transfected bone marrow cells in culture, or the conditioned medium alone is injected into tissue associated with ischemia or delivered by injection into the blood stream, such as an artery supplying an ischemic tissue, or any other artery or vein, the resulting angiogenesis is greater than is achieved by injection of transfected bone marrow stem cells alone.

One of the angiogenesis-promoting factors that most likely participate in initiating angiogenesis in response to ischemia is HIF-1, a potent transcription factor that binds to and stimulates the promoter of several genes involved in responses to hypoxia. Induction and activation of HIF-I is tightly controlled by tissue pO₂; HIF-1 expression increases exponentially as pO₂ decreases, thereby providing a positive feedback loop by which a decrease in pO₂ causes an increase in the expression of gene products that serve as an adaptive response to a low oxygen environment. Activation of HIF-1 leads, for example, to the induction of erythropoietin, genes involved in glycolysis, and to the expression of VEGF. It probably also modulates the expression of many other genes that participate in the adaptive response to low pO² levels. The mechanism by which HIF-1 regulates levels of proteins involved in the response to hypoxia is through transcriptional regulation of genes responding to low pO². Thus, such genes have short DNA sequences within the promoter or enhancer regions that contain HIF-1 binding sites, designated as hypoxia responsive elements (HRE). HIF-1 is a heterodimer with a basic helix-loop-helix motif, consisting of the subunits HIF-1α and HIF-1β. Its levels are regulated by pO2 both transcriptionally and posttranscriptionally—HIF-1 induction is increased by hypoxia, and its half-life is markedly reduced as p² levels increase.

It is relevant that while expression of HIF-1 (as determined in HeLa cells) is exponentially and inversely related to pO₂, the inflection point of the curve occurs at an oxygen saturation of 5%, with maximal activity at 0.5% and /2 maximal activity at 1.5-2.0%. These are relatively low levels of hypoxia, and it is not clear whether such levels occur in the presence of mild levels of myocardial or lower limb ischemia—i.e., levels present in the absence of tissue necrosis (myocardial infarction, and leg ulcerations, respectively). Thus, bone marrow cells could have the capacity to secrete angiogenic factors and thereby enhance collateral development. However, it is possible that such activity may not become manifest in the specific tissue environments treated unless some additional stimulus is present. It is, therefore, a preferred aspect of the invention to co administer, if necessary, bone marrow or bone marrow cell implant with HIF-1. It is anticipated that HIF-1 will provide optimal expression of many of the hypoxia-inducible angiogenic genes present in the bone marrow implant. The HIF-1 can be injected either as the protein, or as the gene. If as the latter, it can be injected either in a plasmid or viral vector, or any other manner that leads to the presence of functionally relevant protein levels.

HIF-1 is a transcription factor that plays a critical role in the transcriptional activation of hypoxia inducible genes. It functions as a heterodimer composed of HIF-1α and HIF-1β subunits. HIF-1 activity is controlled by the stability of the HIF-1α subunit. Thus, HIF-1α is ubiquitinated under normoxic conditions, which targets the molecule for proteasomal degradation. Hypoxia leads to decreased ubiquitination, and therefore greater protein stability. This enhances heterodimer formation and therefore increases HIF-1 activity. The fact that the functional activity of HIF-1 is tightly and inversely coupled to oxygen levels indicates its critical role as a molecular sensor of oxygen, and thereby in modulating the adaptive responses of cells to hypoxia. It has been discovered that the transcriptional activity of HIF-1 derives from the capacity of the heterodimer, which as noted forms only under hypoxic conditions, to bind to a specific DNA hypoxia-responsive recognition element (HRE) present in the promoter of many genes involved in the response of the cell to hypoxia, including VEGF, VEGFR1, VEGFR2, Ang-2, Tie-1, and nitric oxide synthase. Thus, HIF-1 plays a pivotal role in coordinating the tissue response to ischemia.

Because of the lability of HIF-1α in the absence of hypoxia, to assure its constitutive activity even under normoxic conditions, a chimeric construct of the HIF-1α gene has been constructed, consisting of the DNA-binding and dimerization domains from HIF-1α and the transactivation domain from herpes simplex virus VP16 protein as described in Example 8 below. The VP16 domain abolishes the ubiquitination site in HIF-1α, and therefore eliminates the proteasomal-mediated degradation of the protein. Thus, the resulting stable levels of HIF-1α lead to constitutive transactivation of the genes targeted by HIF-1.

It is emphasized, however, that HIF-1 is used as an example of an intervention that could enhance production of angiogenic substances by bone marrow. This invention also covers use of other angiogenic agents, which by enhancing HIF-1 activity (i.e., prolonging its half-life), or by producing effects analogous to HIF-1, stimulate the bone marrow to increase expression of angiogenic factors. A similar approach involves the exposure of autologous bone marrow to endothelial PAS domain protein 1 (EPAS1). EPAS1 shares high structural and functional homology with HIF-1 and is also known as HIF-2.

In another embodiment according to the invention, to enhance VEGF promoter activity, by HIF-1, bone marrow cells can be exposed ex-vivo in culture to hypoxia or other forms of energy, such as, for example, ultrasound, RF, or electromagnetic energy. This intervention increases VEGF and other gene expression. By this effect it may augment the capacity of bone marrow to stimulate angiogenesis. Thus, in this embodiment, the invention involves the ex-vivo stimulation of aspirated autologous bone marrow by HIF-1 (or products that augment the effects of HIF-1 or produce similar effects to HIF-1 on bone marrow) or direct exposure of bone marrow to hypoxic environment followed by the delivery of activated bone marrow cells or media derived from these cells while the cells grow in culture, to the ischemic myocardium or peripheral organ (e.g., ischemic limb) to enhance collateral-dependent perfusion in cardiac and/or peripheral ischemic tissue.

Current data indicate the importance of monocyte-derived cytokines for enhancing collateral function. Monocytes are activated during collateral growth in vivo, and monocyte chemotactic protein-1 (MCP-1) is upregulated by shear stress in vitro. It has been shown that monocytes adhere to the vascular wall during collateral vessel growth (arteriogenesis) and capillary sprouting (angiogenesis). MCP-1 was also shown to enhance collateral growth after femoral artery occlusion in the rabbit chronic hind limb ischemia model (Ito et al., Circ Res 1997; 80:829-3). Activation of monocytes seems to play an important role in collateral growth as well as in capillary sprouting. Increased monocyte recruitment by LPS is associated with increased capillary density as well as enhanced collateral and peripheral conductance at 7 days after experimental arterial occlusion (Arms M. et al., J Clin Invest 1998; 101:40-50.).

A further aspect of the invention involves the ex-vivo stimulation of aspirated autologous bone marrow by MCP-1, followed by the direct delivery of activated bone marrow cells or media derived from these cells while the cells grow in culture, to the ischemic myocardium or peripheral organ (e.g., ischemic limb) to enhance collateral-dependent perfusion and muscular function in cardiac and/or peripheral ischemic tissue. The stimulation of the bone marrow could be by the direct exposure of the bone narrow to MCP-1 in the form of the protein, or the bone marrow cells can be transfected with a vector carrying the MCP-1 gene. For example, bone marrow, or early attaching cells derived from bone marrow, can be transfected with a plasmid vector, or with an adenoviral vector, carrying the MCP-1 transgene.

Granulocyte-macrophage colony-stimulating factor (GM-CSF) and Granulocyte-Colony Stimulatory Factor (G-CSF) are stimulatory cytokines for monocyte maturation and are multipotent hematopoietic growth factors, which are utilized in clinical practice for various hematological pathologies, such as depressed white blood cell count (i.e., leukopenia or granulocytopenia or monocytopenia) which occurs usually in response to immunosuppressive or chemotherapy treatment in cancer patients. GM-CSF has also been described as a multilineage growth factor that induces in vitro colony formation from erythroid burst-forming units, eosinophil colony-forming units (CSF), and multipotential (CSF), as well as from granulocyte-macrophage CSF and granulocyte CFU. (Bot F. J., Exp Hemato 1989, 17:292-5). Ex-vivo exposure to GM-CSF has been shown to induce rapid proliferation of CD-34⁺ progenitor cells. (Egeland T. et al., Blood 1991; 78:3192-g.) These cells have the potential to differentiate into vascular endothelial cells and may naturally be involved in postnatal angiogenesis. In addition, GM-CSF carries multiple stimulatory effects on macrophage/monocyte proliferation, differentiation, motility and survival (reduced apoptotic rate). Consistent with the combined known effects on bone marrow derived endothelial progenitor cells and monocytes, it is another aspect of the invention to use GM-CSF as an adjunctive treatment to autologous bone marrow injections aimed to induce new blood vessel formation and differentiation in ischemic cardiovascular organs. Moreover, GM-CSF may further enhance therapeutic myocardial angiogenesis caused by bone marrow, by augmenting the effect of bone marrow, or by further stimulating, administered either in vivo or in vitro, bone marrow that is also being stimulated by agents such as HIF-1, EPAS 1, hypoxia, or MCP-1.

However, bone marrow cells that are injected into regions in which collateral blood vessel development is desired in order to enhance the delivery of blood to ischemic regions may not produce optimal angiogenic effects when certain “at risk” conditions prevail. For example, there is evidence demonstrating that angiogenesis is impaired in the presence of hypercholesterolemia, and it is also compromised with aging. In addition, there are a number of genetic and other disorders impairing naturally occurring angiogenic processes as compared with that found in normal young healthy individuals. It has also now been discovered that the function of bone marrow cells is also compromised in the presence of hypercholesterolemia with aging, and by such genetic and other disorders that impair naturally occurring angiogenic processes as compared with that found in normal young healthy individuals.

Hypercholesterolemia is a dominantly inherited genetic condition that results in markedly elevated low-density lipoprotein cholesterol levels beginning at birth, and resulting in myocardial infarctions at an early age. “Aging” as the term is used herein is not necessarily measured in years, but is measured in terms of deterioration of the body's ability to maintain the vascular system in a healthy condition. Nevertheless, the ability of the body to maintain vasular health tends to deteriorate with time (i.e., with age) as well.

Experimental evidence suggests collateral development of the vasculature is impaired in the elderly, who represent the largest cohort of patients affected by advanced arteriosclerosis. Both the functions of bone marrow progenitor cells (BMPCs) and HIF-1 activity are reduced with aging. Therefore, all of the age-related factors that impair collateral development would also affect the bone marrow-derived progenitor cells, such as bone marrow-derived stromal cells (MSCs), retrieved from older patients and delivered to their ischemic tissue. It follows that older patients have impaired collateral formation in part due to impaired HIF-related mechanisms, and that exposing developing collaterals to increased concentrations of HIF-1-induced cytokines will augment collateral formation.

In another aspect, the present invention recognizes the confounding effects of these and other “risk factors”, and describes throughout this application methods that are designed to enhance the angiogenic potential of such functionally compromised bone marrow cells by transducing these cells with polynucleotides encoding proteins that will enhance the capacity of such impaired bone-marrow cells to foster development of collateral blood vessels. For example, Example 9 and FIG. 9 describe the enhanced production in vitro of recombinant VEGF by MSCs transfected with an adenoviral vector encoding HIF-1α/VP 16 obtained from coronary artery disease patients.

There are several additional genes whose protein products importantly enhance the capacity of compromised bone marrow cells to enhance collateral blood vessel formation. For example, in another embodiment, the invention provides methods for transforming bone marrow cells with a gene encoding one or species of nitric oxide synthase (NOS). A “NOS gene” or a “polynucleotide encoding NOS” as the term is used herein means any of the known isoforms of NOS, including inducing NOS (iNOS) and endothelial NOS (eNOS), as well as NOS genes that have been mutated such that the magnitude of their expression is altered, or that they encode an altered protein, either of which results in a more potent angiogenic effect.

The rationale for transducing cells with a polynucleotide encoding NOS is based on the fact that VEGF, one of the more potent angiogenic agents identified, works through NOS signaling pathways. For example, it has been shown that VEGF fails to induce angiogenesis in mice in which NOS gene has been knocked out. Moreover, nitric oxide (NO), the protein product of NOS, has multiple actions that induce angiogenesis and, moreover, induce the expression of many different genes, many of which are involved in angiogenesis. Thus, transfecting bone marrow cells with NOS, augments the intrinsic capacity of bone marrow cells to secrete multiple angiogenic cytokines and growth factors and also stimulates expression of multiple angiogenesis-related genes. The invention also provides such NOS-transfected bone marrow cells, especially ABM cells, or media derived from these cells while the cells grow in culture.

Another family of genes this invention describes as having the capacity to augment the potential of bone marrow cells to enhance collateral blood vessel development is the fibroblast growth factor (FGF) family. This family of genes involves over fourteen closely related genes including, but not limited to, FGF 1, FGF 2, FGF 4, and FGF 5. The rationale for transducing bone marrow cells with one of the genes in the FGF family is that FGF is known to be a potent stimulator of angiogenesis, and also is capable of stimulating the expression of multiple genes, many of whose proteins products are also capable of inducing angiogenesis.

Thus, in yet another embodiment, the invention provides a method for using bone marrow cells transfected with a polynucleotide encoding one of the FGF family of peptides to enhance the capacity of bone marrow cells to increase development of collateral blood vessel development, such as bone marrow cells that may have an impaired capacity to enhance angiogenesis because of diverse risk factors, including but not limited to hypercholesterolemia and aging. The invention also provides such FGF-transfected bone marrow cells, especially ABM cells, or media derived from these cells while the cells grow in culture.

PR39, another gene expressed by monocytes/macrophages, is another gene that this invention describes as being able to enhance the angiogenic potential of bone marrow cells to improve collateral formation. The rationale for transducing bone marrow cells with the gene encoding this protein derives from the fact that PR39 inhibits the proteasomal degradation of HIF-1α, resulting in accelerated formation of vascular structures in vitro and increased myocardial vasculature in mice. By increasing the steady state levels of HIF-1α, the heterodimer—HIF-1α/HIF-1β—forms, which is a transcription factor that induces the expression of HIF-1-related genes. The protein products of many of these genes promote the development of angiogenesis. The rationale for this strategy-increasing the steady-state levels of HIF-1α—has been described in detail above. In still another embodiment, the invention also provides such PR39-transfected bone marrow cells, especially ABM cells, or media derived from these cells while the cells are grown in culture.

For example, ABM cells collected from a subject can be transfected, ex vivo, with a plasmid vector, or with an adenoviral vector, carrying an angiogenic cytokine growth factor or mammalian angiogenesis promoting factor transgene, such as the HIF-1 or EPAS 1 transgene, or a transgene encoding PR39, or a member of the NOS or FGF families, for expression thereof in the cells and/or in the subject when the transfected cells are injected into a treatment site as described herein, or media derived from these cells while the cells are grown in culture is injected into a treatment site.

However, fresh bone marrow or bone marrow cells in solution can be difficult to transfect with a vector encoding the therapeutic cytokines, growth factors and angiogenesis-promoting factors described herein. To overcome this difficulty, it has been discovered that the efficiency with which bone marrow cells can be transfected ex vivo (e. g. in culture) with a vector carrying a transgene is greatly enhanced when bone marrow cells are grown in a container for a sufficient period of time to allow adherence of early attaching cells from the bone marrow to the container and the early attaching cells are selected for the transfection. “Early attaching cells” as the term is used herein means the cells from the culture medium containing bone marrow, or from bone marrow cells seeded into the container, that do not wash away after growth at suitable culture conditions for about 8 hours (e.g., overnight) to about 24 hours. The early attaching cells are mostly monocytes, endothelial precursor cells, or other hematopoietic lineage cells. Inoculation takes place after culture of the cells for a period of several hours, and the inoculated cells begin to produce the transgene products after about 12 hours to 3 days. The early attaching cells can be inoculated with a vector encoding one or more angiogenic cytokines, growth factors and/or factors that promote angiogenesis in mammalian cells by any method known in the art, for example by in vitro contact for a period of about 2 hours to about 3 days after the inoculation. The vector used can be selected from any of those known in the art and include, but without limitation thereto, those described herein. The vector (e.g. a virus or plasmid) is generally washed out about 2 hours to about 3 days after the inoculation before the cells are prepared for administration to the patient.

Optionally, the ABM can be filtered prior to placement in the culture to remove particles larger than about 300μ to about 200μ. Bone marrow cells can also be separated from the filtered ABM for growth in the container leading to production of early attaching cells. Suitable culture conditions are well known in the art and include, but are not limited to, those described in the Examples herein.

Suitable transgenes for transfecting bone marrow early attaching cells according to the invention methods include, but without limitation thereto, those encoding such angiogenesis-promoting agents as HIF-1, EPAS 1 (also known as HIF-2), MCP-1, CM-CSF, NOS, FGF, and the like. An effective amount of the transfected early attaching cells derived from bone marrow prepared as described herein can be directly administered to (i.e. injected into) a desired site in a patient to enhance collateral blood vessel formation at the site in the patient. Particularly effective sites for administration of cells transfected with an angiogenesis-promoting agent include heart muscle or skeletal muscle, such as in the leg, to enhance collateral-dependent perfusion in cardiac and/or peripheral ischemic tissue. The cells or media derived from such cells can also be injected into the vascular system so that they are delivered to the desired site by the blood.

In non-limiting illustration of the invention methods for obtaining enhanced transfection efficiency of bone marrow cells, studies have been conducted utilizing the X-gal transgene in an adenovirus vector to transfect bone marrow early attaching cells prepared as described above. In these studies, staining of transfected cells with X-gal a suitable period of time after transfection shows that, compared with non-adherent bone marrow cells or fresh bone marrow, susceptibility of bone marrow early attaching cells to transfection is substantially increased.

The polynucleotide encoding the therapeutic protein may be “functionally appended to”, or “operatively associated with”, a signal sequence that can “transport” the encoded product across the cell membrane. A variety of such signal sequences are known and can be used by those skilled in the art without undue experimentation.

Gene transfer vectors (also referred to as “expression vectors”) contemplated for such purposes are recombinant nucleic acid molecules that are used to transport nucleic acid into host cells for expression and/or replication thereof. Expression vectors may be either circular or linear, and are capable of incorporating a variety of nucleic acid constructs therein. Expression vectors typically come in the form of a plasmid that, upon introduction into an appropriate host cell, results in expression of the inserted nucleic acid.

Suitable viral vectors for use in gene therapy have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, Bio Techniques 7:980-990, 1992; Anderson et al., Nature 392:25-30 Suppl., 1998; Verma and Somia, Nature 389:239-242, 1997; Wilson, New Engl. J Med. 334:1185-1187 (1996), each of which is incorporated herein by reference). Preferred gene transfer vectors are replication-deficient adenovirus carrying the cDNA to effect development of collateral arteries in a subject suffering progressive coronary occlusion (Barr et al., “PCGT Catheter-Based Gene Transfer Into the Heart Using Replication-Deficient Recombinant Adenoviruses,” Journal of Cellular Biochemistry, Supplement 17D, p. 195, Abstract P101 (March 1993); Barr et al., “Efficient catheter-mediated gene transfer into the heart using replication-defective adenovirus,” Gene Therapy, vol. 1:51-58 (1994)). In general, the gene of interest may be transferred to the heart (or skeletal muscle), including cardiac myocytes (and skeletal myocytes), in vivo and direct constitutive production of the encoded protein.

Several different gene transfer approaches are feasible, including the helper-independent replication deficient human adenovirus 5 system. The recombinant adenoviral vectors based on the human adenovirus 5 (Virology 163:614-617, 1988) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in tissue/cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells well.

The amount of exogenous nucleic acid introduced into a host organism, cell or cellular system can be varied by those of skill in the art according to the needs of the individual being treated. For example, when a viral vector is employed to achieve gene transfer, the amount of nucleic acid introduced to the cells to be transfected can be varied by varying the amount of plaque forming units (PFU) of the viral vector.

In yet another embodiment according to the invention, there are provided methods for enhancing collateral blood vessel formation in a subject in need thereof by obtaining ABM from the patient; growing the ABM under suitable culture conditions in a container for a period of time sufficient to promote production by the bone marrow of early attaching cells, which early attaching cells adhere to the container. The early attaching cells are transfected in culture as described above (i.e. in vitro) with a vector as described herein comprising a polynucleotide that encodes one or more agents selected from angiogenic cytokines, growth factors and mammalian angiogenesis-promoting factors, and the like, and the processed (i.e., transfected) early attaching cells (and/or medium in which they are cultured after transfection) are then directly administered to a desired site in the patient so as to deliver to the site the expressed agent(s). For example, in one embodiment, the angiogenesis-promoting agent can be transiently expressed in the subject into which the transfected cells are injected, thus delivering the therapeutic angiogenesis-promoting agents, or a combination thereof, to the ischemic site and leading to enhanced collateral blood vessel formation at the site of administration in the patient.

The phrase “marrow-derived stromal cells” as used herein means CD34 minus/CD45 minus early attaching cells that can be obtained from a sample of bone marrow.

As used herein, the phrase “transcription regulatory region” refers to that portion of a nucleic acid or gene construct that controls the initiation of mRNA transcription. Regulatory regions contemplated for use herein, in the absence of the non-mammalian transactivator, typically comprise at least a minimal promoter in combination with a regulatory element responsive to the ligand/receptor peptide complex. A minimal promoter, when combined with a regulatory element, functions to initiate mRNA transcription in response to a ligand/functional dimer complex. However, transcription will not occur unless the required inducer (ligand therefor) is present. However, as described herein certain of the invention chimeric protein heterodimers activate or repress mRNA transcription even in the absence of ligand for the DNA binding domain.

As used herein, the phrase “operatively associated with” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and promoter such that an RNA polymerase that specifically recognizes, binds to and transcribes the DNA initiates transcription of such DNA from the promoter.

Preferably, the transcription regulatory region further comprises a binding site for ubiquitous transcription factor(s). Such binding sites are preferably positioned between the promoter and the regulatory element. Suitable ubiquitous transcription factors for use herein are well known in the art and include, for example, Sp1.

Exemplary eukaryotic expression vectors include eukaryotic constructs, such as the pSV-2 gpt system (Mulligan et al., (1979) Nature, 277:108-114); PBLUESKRIPT® vector (Stratagene, La Jolla, Calif.), the expression cloning vector described by Genetics Institute (Science, (1985) 228:810-815), and the like. Each of these plasmid vectors is capable of promoting expression of the protein of interest.

In a specific embodiment, a gene transfer vector contemplated for use herein is a naked plasmid, a viral vector, such as Adenovirus, adeno-associated virus, a herpes-simplex virus based vector, a synthetic vector for gene therapy, and the like (see, e.g., Suhr et al., Arch. of Neurol. 50:1252-1268, 1993). For example, a gene transfer vector employed herein can be a retroviral vector. Retroviral vectors contemplated for use herein are gene transfer plasmids that have an expression construct containing an exogenous nucleic acid residing between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764).

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, each of which is hereby incorporated herein by reference, in its entirety. These documents provide a description of methods for efficiently introducing nucleic acids into human cells using such retroviral vectors. Other retroviral vectors include, for example, mouse mammary tumor virus vectors (e.g., Shackleford et al., (1988) PNAS, USA, 85:9655-9659), human immunodeficiency virus (e.g., Naldini et al. (1996) Science 272:165-320), and the like.

Various procedures are also well known in the art for providing helper cells that produce retroviral vector particles that are essentially free of replicating virus. See, for example, U.S. Pat. No. 4,650,764; Miller, Human Gene Therapy, 1:5-14, 1990; Markowitz, et al., Journal of Virology, 61(4): 1120-1124, 1988; Watanabe, et al., Molecular and Cellular Biology, 3(12):2241-2249, 1983; Danos, et al., PNAS, 85:6460-6464, 1988; and Bosselman, et al., Molecular and Cellular Biology, 7(5): 1797-1806, 1987, which disclose procedures for producing viral vectors and helper cells that minimize the chances for producing a viral vector that includes a replicating virus.

Recombinant retroviruses suitable for prepackaging with polynucleotides that encode therapeutic proteins, such as angiogenic growth factors, are produced employing well-known methods for producing retroviral virions. See, for example, U.S. Pat. No. 4,650,764; Miller, supra 1990; Markowitz, et al., supra 1988; Watanabe, et al., supra 1983; Danos, et al., PNAS, 85:6460-6464, 1988; and Bosselman, et al., Molecular and Cellular Biology, 7(5):1797-1806, 1987.

In the examples below, certain testing illustrating aspects of the invention is set forth. These examples are non-limitative.

### EXAMPLES

### Example 1

Effect of Bone Marrow Cultured Media-on Endothelial Cell Proliferation

Studies were conducted to determine whether aspirated pig autologous bone marrow cells obtained secreted VEGF, a potent angiogenic factor, and MCP-1, which recently has been identified as an important angiogenic co-factor. Bone marrow was cultured in vitro for four weeks. The conditioned medium was added to cultured pig aortic endothelial cells (PAECs), and after four days proliferation was assessed. VEGF and MCP-1 levels in the conditioned medium were assayed using ELISA. During the four weeks in culture, BM cells secreted VEGF and MCP-1, such that their concentrations increased in a time-related manner. The resulting conditioned medium enhanced, in a dose-related manner, the proliferation of PAECs. The results indicate that BM cells are capable of secreting potent angiogenic cytokines such as VEGF and MCP-1 and of inducing proliferation of vascular endothelial cells.

Pig Bone Marrow Culture

Bone marrow (BM) cells were harvested under sterile conditions from pigs with chronic myocardial ischemia in preservative free heparin (20 units/ml BM cells) and filtered sequentially using 300[t and 200p stainless steel mesh filters. BM cells were then isolated by Ficoll-Hypaque gradient centrifugation and cultured in long-term culture medium (LTCM) (Stem Cell Tech, Vancouver, British Columbia, Canada) at 330° C. with 5% CO₂ in T-25 culture flask. The seeding density of the BMCs in each culture was 7×10⁶/ml. Weekly, one half of the medium was removed and replaced with fresh LTCM. The removed medium was filtered (0.2μ filter) and stored at −200° C. for subsequent Enzyme-linked Immunosorbent Assay (ELISA) and cell proliferation assays.

Isolation and Culture of Pig Aortic Endothelial Cells

Fresh pig aortic endothelial cells (PAECs) were isolated using conventional methods. Endothelial cell growth medium (EGM-2 medium, Clonetics, San Diego, Calif.), containing 2% FBS, hydrocortisone, human FGF, VEGF, human EGF, IGF, heparin and antibiotics, at 37° C. with 5% carbon dioxide. When the cells became confluent at about 7 days, they were split by 2.5% trypsin and cultured thereafter in medium 199 with 10% FBS. Their identity was confirmed by typical endothelial cell morphology and by immunohistochemistry staining for factor VIII. Passage 3-10 was used for the proliferation study.

Effects of Conditioned Medium on Aortic Endothelial Cells

Cell proliferation assay: PAECs (Passage 3-10) were removed from culture flasks by trypsinization. The detached cells were transferred to 96-well culture plates and plated at a seeding density of 5,000 cells/well. Cells were cultured for 2-3 days before being used in proliferation and DNA synthesis experiments. The conditioned medium of BM cells cultures were collected at 4 weeks, medium from 7 culture flasks were pooled and used in the bioassay. Aliquotes (10 μL, 30 μL, 100 μL or 200 μL) of pooled conditioned medium, or LTCM (200 μL, as control), were added to confluent PAECs in 96-well plates in triplicate. Four days following culture with conditioned medium or control medium, the PAECs were trypsinized and counted using a cell counter (Coulter Counter Beckman Corporation, Miami Fla.).

Effects of Conditioned Medium on PAEC DNA Synthesis

Aliquots (10 μL, 30 μL, 100 μL or 200 μL) of conditioned medium from pooled samples or control medium (LTCM, 200 μL) were added to PAECs in 96-well plate (same seeding density as above) in triplicate. After 2 days, 1 μCi tritiated thymidine was added to each well. Forty-eight hours later, DNA in PAECs was harvested using a cell harvester (Mach III M Tomtec, Hamden, Conn.) and radioactivity was counted by liquid scintillation counter (Multi-detector Liquid Scintillation Luminescence Counter EG&G Wallac, Turku, Finland).

Determination of VEGF and MCP-1 in Conditioned Medium by ELISA VEGF

The concentration of VEGF in conditioned medium was measured using a sandwich ELISA kit (Chemicon International Inc., Temecula, Calif.). Briefly, a plate pre-coated with anti-human VEGF antibody was used to bind VEGF in the conditioned medium or to a known concentration of recombinant VEGF. The complex was detected by the biotinylated anti-VEGF antibody, which binds to the captured VEGF. The biotinylated VEGF antibody in turn was detected by streptavidin-alkaline phosphatase and color generating solution. The anti-human VEGF antibody cross-reacts with porcine VEGF.

Determination of MCP-1 in Conditioned Medium by ELISA

The concentration of MCP-1 in conditioned medium was assayed by sandwich enzyme immunoassay kit (R &D Systems, Minneapolis, Minn.): a plate pre-coated with anti human MCP-1 antibody was used to bind MCP-1 in the conditioned medium or to a known concentration of recombinant protein. The complex was detected by the biotinylated anti-MCP-1 antibody, which binds to the captured MCP-1. The biotinylated MCP-1 antibody in turn was detected by streptavidin-alkaline phosphatase and color generating solution. The anti-human MCP-1 antibody cross-reacts with porcine MCP-1.

Results

The BM conditioned medium collected at four weeks increased, in a dose-related manner, the proliferation of PAECs (FIG. 1). This was demonstrated by counting the number of cells directly and by measuring tritiated thymidine uptake (p<0.001 for both measurements). The dose-related response demonstrated a descending limb; proliferation decreased with 200 μL conditioned medium compared to 30 μL and 100 μL (P=0.003 for both comparisons). Similar dose-related results were observed in the tritiated thymidine uptake studies (P=0.03 for 30 μL and 100 μL compared to 200 μL, respectively).

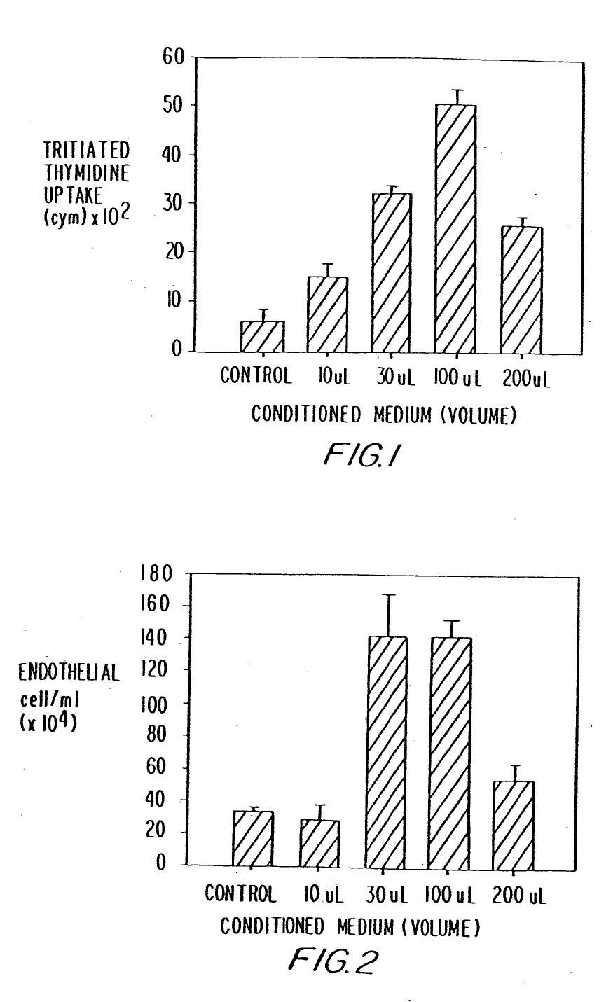

A limited number (5±4%) of freshly aspirated BM cells stained positive for factor VIII. The results are set forth in FIG. 2. This contrasted to 57±14% of the adherent layer of BM cells cultured for 4 weeks, of which 60±23% were endothelial-like cells and 40±28% appeared to be megakaryocytes.

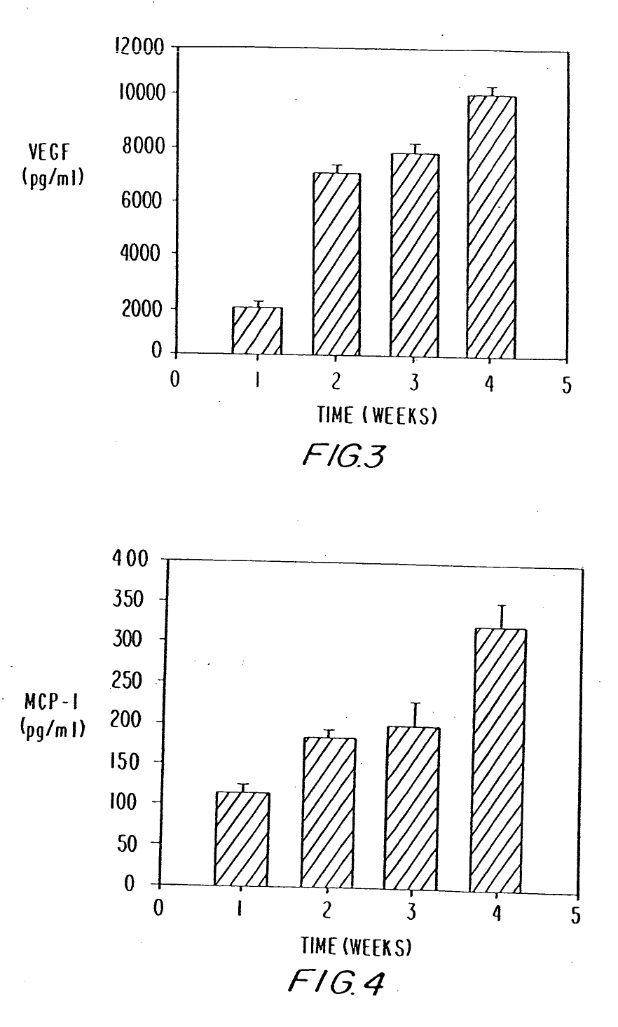

Over a 4-week period, the concentrations of VEGF and MCP-1 in the BM conditioned medium increased gradually to 10 and 3 times the 1st week level, respectively (P<0.001 for both comparisons) (FIG. 3). In comparison, VEGF and MCP-1 levels in a control culture medium, not exposed to BM, were 0 and 11±2 pg/ml, respectively, as shown in FIG. 4.

### Example 2

Effects of Hypoxia on VEGF Secretion by Cultured Pig Bone Marrow Cells

It was demonstrated that hypoxia markedly increases the expression of VEGF by cultured bone marrow endothelial cells, results indicating that ex-vivo exposure to hypoxia, by increasing expression of hypoxia-inducible angiogenic factors, can further increase the collateral enhancing effect of bone marrow cells and its conditioned media to be injected in ischemic muscular tissue. Pig bone marrow was harvested and filtered sequentially using 300μ and 200μ stainless steel mesh filters. BMCs were then isolated by Ficoll-Hypaque gradient centrifugation and cultured at 33° C. with 5% CO₂ in T-75 culture flasks. When cells became confluent at about 7 days, they were split 1:3 by trypsinization. After 4 weeks of culture, the BMCs were either exposed to hypoxic conditions (placed in a chamber containing 1% oxygen) for 24 to 120 hrs, or maintained under normal conditions. The resulting conditioned medium was collected and VEGF, MCP-1 were analyzed by ELISA.

Exposure to hypoxia markedly increased VEGF secretion: At 24 hours VEGF concentration increased from 106±13 pg/ml under normoxic, to 1,600±196 pg/ml under hypoxic conditions (p=0.0002); after 120 hours it increased from 4,163±62 to 6,028±167 pg/ml (p<0.001). A separate study was performed on freshly isolated BMCs, and the same trend was found. Hypoxia also slowed the rate of proliferation of BMCs. MCP-1 expression was not increased by hypoxia, a not unexpected finding as its promoter is not known to have HIF binding sites.

### Example 3

Effect of Bone Marrow Cultured Media on Endothelial Cell Tube Formation

It was demonstrated, using pig endothelial cells and vascular smooth muscle cells co-culture technique, that the conditioned medium of bone marrow cells induced the formation of structural vascular tubes in vitro. No such effect on vascular tube formation was observed without exposure to bone marrow conditioned medium. The results suggest that bone marrow cells and their secreted factors exert pro-angiogenic effects.

### Example 4

The effect of Transendocardial Delivery of Autologous Bone Marrow on Collateral Perfusion and Regional Function in Chronic Myocardial Ischemia Model

Chronic myocardial ischemia was created in 14 pigs by the implantation of ameroid constrictors around the left circumflex coronary artery. Four weeks after implantation, 7 animals underwent transendocardial injections of freshly aspirated ABM into the ischemic zone using a transendocardial injection catheter (2.4 ml per animal injected at 12 sites) and 7 control animals were injected with heparinized saline. At baseline and 4 weeks later, animals underwent rest and pacing echocardiogram to assess regional contractility (% myocardial thickening), and microsphere study to assess collateral-dependent perfusion at rest and during adenosine infusion. Four weeks after injection of ABM collateral flow (expressed as the ratio of ischemic/normal zone×100) improved in ABM-treated pigs but not in controls (ABM: 95±13 vs. 81±11 at rest, P-0.017; 85±19 vs. 72±10 during adenosine, P=0.046; Controls: 86±14 vs. 86±14 at rest, P=NS; 73±17 vs. 72±14 during adenosine, P=0.63). Similarly, contractility increased in ABM-treated pigs but not in controls (ABM: 83±21 vs. 60±32 at rest, P=0.04; 91±44 vs. 35±43 during pacing, P=0.056; Controls: 69±48 vs. 64±46 at rest, P=0.74; 65±56 vs. 37±56 during pacing, P=0.23).

The results indicate that catheter-based transendocardial injection of ABM can augment collateral perfusion and myocardial function in ischemic myocardium, findings suggesting that this approach may constitute a novel therapeutic strategy for achieving optimal therapeutic angiogenesis.

Fourteen specific-pathogen-free domestic pigs weighing approximately 70 kg were anesthetized, intubated, and received supplemental O₂ at 2 L/min as well as 1-2% isoflurene inhalation throughout the procedure. Arterial access was obtained via right femoral artery isolation and insertion of an 8 French sheath. The left circumflex artery was isolated through a left lateral thoracotomy and a metal encased ameroid constrictor was implanted at the very proximal part of the artery. Four weeks after the ameroid constrictor implantation all pigs underwent (1) a selective left and right coronary angiography for verification of ameroid occlusion and assessment of collateral flow; (2) transthoracic echocardiography studies; and (3) regional myocardial blood flow assessment.

Bone Marrow Aspiration and Preparation and Intramyocardial Injection

Immediately after completion of the baseline assessment, all animals underwent BM aspiration from the left femoral shaft using standard techniques. BM was from aspirated 2 sites (3 ml per site) using preservative free heparinized glass syringes (20 unit heparin/1 ml fresh BM). The aspirated bone marrow was immediately macro-filtered using 300μ and 200μ stainless steel filters, sequentially. Then, the bone marrow was injected using a transendocardial injection catheter into the myocardium in 12 sites (0.2 ml per injection site for total of 2.4 ml) directed to the ischemic myocardial territory and its borderline region.

MSCs Injected into the Region of Developing Collaterals Express Collateral-Enhancing-Related Cytokines in vivo and Incorporate into Developing Collaterals.

Transthoracic echocardiography images of short and long axis views at the mid-papillary muscle level were recorded in animals at baseline and during pacing, at baseline and during follow-up evaluation at four weeks after ABM implantation. Fractional shortening measurements were obtained by measuring the % wall thickening (end-systolic thickness minus end-diastolic thickness/end-diastolic thickness)×100. Those measurements were taken from the ischemic territory (lateral area) and remote territory (anterior-septal area). Subsequently, a temporary pacemaker electrode was inserted via a right femoral venous sheath and positioned in the right atrium. Animals were paced at 180/minute for 2 minutes and echocardiographic images were simultaneously recorded.

Regional Myocardial Blood Flow

Regional myocardial blood flow measurements were performed at rest and during maximal coronary vasodilation by use of multiple fluorescent colored microspheres (Interactive Medical Technologies, West Los Angeles, Calif.) and quantified by the reference sample technique (Heymann M A, et al., Prog Cardiovasc Dis 1977; 20:55-79). Fluorescent microspheres (0.8 ml, 5×106 microspheres/ml, 15 μm diameter in a saline suspension with 0.01% Tween 80) were injected into the left atrium via a 6F Judkins left 3.5 diagnostic catheter. Maximal coronary vasodilation was induced by infusing adenosine at a constant rate of 140 μg/kg/min (Fujisawa USA, Deerfield, Ill.) into the left femoral vein over a period of 6 minutes. During the last 2 minutes of the infusion, microsphere injection and blood reference withdrawal were undertaken in identical fashion to the rest study.

Following completion of the perfusion assessment, animals were sacrificed with an overdose of sodium pentobarbital and KCL. Hearts were harvested, flushed with Ringer Lactate, perfusion-fixed for 10-15 minutes, and subsequently immersion-fixed with 10% buffered formaldehyde for 3 days. After fixation was completed, the hearts were cut along the short axis into 7-mm thick slices. The 2 central slices were each divided into 8 similar sized wedges, which were further cut into endocardial and epicardial subsegments. The average of 8 lateral ischemic zone and 8 septal normal zone sub-segments measurements were used for assessment of endocardial and epicardial regional myocardial blood flow. The relative collateral flow was also computed as the ratio of the ischemic zone/non ischemic zone (IZ/NIZ) blood flow.

Histopathology

To assess whether injecting BM aspirate via the use of an injection catheter was associated with mechanical cell damage, standard BM smears were prepared before and after propelling the freshly filtered ABM aspirate through the needle using similar injecting pressure as in the in-vivo study. Morphological assessment was performed by an independent experienced technician who was blinded to the study protocol.

Histopathology assessment was performed on sampled heart tissue. In the pilot study, 7-mm thick short-axis slices were examined under UV light to identify fluorescent-tagged areas. Each identified area was cut into 3 full thickness adjacent blocks (central, right and left) that were immersion-fixed in 10% buffered formaldehyde. Subsequently, each such block was cut into 3 levels, of which 2 were stained with Hematoxylin and Eosin (H&E) and one with PAS. In addition, one fresh fluorescent-labeled tissue block was obtained from the ischemic region of each animal and was embedded in OCT compound (Sakura Finetek USA Inc., Torrance, Calif.) and frozen in liquid nitrogen. Frozen sections of these snap-frozen myocardial tissues were air dried and fixed with acetone. Immunoperoxidase stain was performed with the automated Dako immunno Stainer (Dako, Carpenteria, Calif.). The intrinsic peroxidase and non-specific uptake were blocked with 0.3% hydrogen peroxidase and 10% ovo-albumin. Monoclonal mouse antibody against CD-34 (Becton Dickinson, San Jose, Calif.) was used as the primary antibody. The linking antibody was a biotinylated goat anti-mouse IgG antibody and the tertiary antibody was strepavidin conjugated with horse reddish peroxidase. Diaminobenzidine (DAB) was used as the chromogen and the sections were counterstained with 1% methylgreen. After dehydration and clearing, the slides were mounted and examined with a Nikon Labphot microscope.

In the efficacy study, full-thickness, 1.5 square centimeter sections from the ischemic and non-ischemic regions were processed for paraffin sections. Each of the samples was stained with H&E, Masson's trichrome, and factor VIII related antigen. The immunoperoxidase stained slides were studied for density of endothelial cell population and vascularization. The latter was distinguished from the former by the presence of a lumen. Vascularity was assessed using 5 photomicrographs samples of the factor VIII stained slides taken from the inner half of the ischemic and non-ischemic myocardium. Density of endothelial cells was assessed using digitized images of the same photomicrographs. The density of the endothelial population was determined by Sigma-Scan Pro morphometry software using the intensity threshold method. The total endothelial area for each sample as well as for each specimen were obtained along with the relative percent endothelial area (endothelial area/area of the myocardium studied). The total endothelial area was also calculated as the relative percent of the non-infarcted (viable) area of the myocardium studied. The trichrome stained sections were digitized and the area occupied by the blue staining collagen as well as the total area of the section excluding the area occupied by the epicardium (which normally contained collagen) were measured using Sigma-Scan Pro. The infarcted area was then calculated as the area occupied by the blue staining.

Procedural Data

Intra-myocardial injections either with ABM or placebo were not associated with any acute change in mean blood pressure, heart rate or induction of arrhythmia. All hemodynamic parameters were comparable between the two groups. Pair-wise comparison showed similar hemodynamic parameters within each group in the index compared to the follow-up procedure except for higher initial mean arterial blood pressure at follow-up in the control group (P=0.03) with no subsequent differences during pacing or adenosine infusion.

Myocardial Function

Regional myocardial function assessment is shown in Table I below. Preintervention relative fractional wall thickening, expressed as ischemic zone to non-ischemic zone (IZ/NIZ) ratio×100, at rest and during pacing, was similar between groups (P=0.86 and 0.96, respectively). At-4 weeks following the intra-myocardial injection of ABM, improved regional wall thickening occurred at rest and during pacing, which was due to an ˜50% increase in wall thickening of the collateral-dependent ischemic lateral wall. No significant changes were observed in the control animals, although a trend towards improvement in wall thickening was noted in the ischemic area during pacing at follow-up.

TABLE 1
Regional Contractility of the Ischemic Wall

| | Baseline | Follow-up | P |
|---|---|---|---|
| Rest | | | |
| ABM (%) | 60 ± 32 | 83 ± 21 | 0.04 |
| Control (%) | 64 ± 46 | 69 ± 48 | 0.74 |
| Pacing | | | |
| ABM (%) | 36 ± 43 | 91 ± 44 | 0.056 |
| Control (%) | 37 ± 56 | 65 ± 56 | 0.23 |

ABM indicates autologous bone marrow.

Myocardial Perfusion Data

Regional myocardial perfusion assessment is shown in Table II below. There were no differences between the, treated and control groups in the pre-intervention relative transmural myocardial perfusion, IZ/NIZ, at rest and during adenosine infusion (P=0.42 and 0.96, respectively). At 4 weeks following ABM injection, relative regional transmural myocardial perfusion at rest and during pacing improved significantly. This was due to an absolute improvement in myocardial perfusion in the ischemic zone both at rest (an increase of 57%, P=0.08) and during adenosine infusion (37%, P=0.09), while no significant changes were noted in absolute flow to the non-ischemic zone either at rest (increase of 35%, P=0.18) or during adenosine infusion (increase of 25%, P=0.26). The increase in regional myocardial blood flow found in the ischemic zones consisted of both endocardial (73%) and epicardial (62%) regional improvement at rest, with somewhat lesser improvement during adenosine infusion (40% in both zones). At 4 weeks, the control group showed no differences in transmural, endocardial or epicardial perfusion in the ischemic and non-ischemic zones compared to pre-intervention values.

TABLE 2
Regional Myocardial Perfusion

| | Baseline | Follow-up | P |
|---|---|---|---|
| Rest | | | |
| ABM (%) | 83 ± 12 | 98 ± 14 | 0.001 |
| Control (%) | 89 ± 9 | 92 ± 0.1 | 0.43 |
| Adenosine | | | |
| ABM (%) | 78 ± 12 | 89 ± 18 | 0.025 |
| Control (%) | 77 ± 5 | 78 ± 11 | 0.75 |

ABM indicates autologous bone marrow.

Histopathology and Vascularity Assessment

Assessment of BM smears before and after passing the filtrated aspirate through the injecting catheter revealed normal structure, absence of macro-aggregates and no evidence of cell fragments or distorted cell shapes. Histopathology at day 1 following injections revealed acute lesions characterized by fibrin and inflammatory tract with dispersed cellular infiltration. The infiltrate was characterized by mononuclear cells that morphologically could not be differentiated from a BM infiltrate. Cellularity was maximal at 3 and 7 days and declined subsequently over time. At 3 weeks, more fibrosis was seen in the 0.5 ml injection-sites compared to the 0.2 ml. CD-34 immunostaining, designed to identify BM-derived progenitor cells, was performed in sections demonstrating the maximal cellular infiltrate. Overall, it was estimated that 4-6% of the cellular infiltrate showed positive immunoreactivity to CD-34.

Small areas of patchy necrosis occupying overall<10% of the examined ischemic myocardium characterized the ischemic territory in both groups. The non-ischemic area revealed normal myocardial structure. Changes in the histomorphometric characteristics of the two groups were compared. There were no differences in the total area occupied by any blood vessel as well as the number of blood vessels>50 μm in diameter. However, comparison of the total areas stained positive for factor VIII (endothelial cells with and without lumen) in the ischemic versus the non-ischemic territories revealed differences between the 2 groups. In the ABM group, the total endothelial cell area in the ischemic collateral-dependent zone was 100% higher than that observed in the nonischemic territory (11.6±5.0 vs. 5.7±2.3% area, P=0.016), whereas there was no significant difference in the control group (12.3±5.5 vs. 8.2±3.1% area, P=0.11). However, other parameters of vascularity, including % area occupied by any blood vessel and number of blood vessels>50 μm were similar in the ischemic and non-ischemic territories in both groups.

### Example 5

The Effect of Autologous bone Marrow Stimulated in vivo by Pre-Administration of GMCSF in Animal Model of Myocardial Ischemia

Chronic myocardial ischemia was created in 16 pigs by the implantation of ameroid constrictors around the left circumflex coronary artery. At four weeks minus 3 days after ameroid implantation, 8 animals underwent subcutaneous injection of GM-CSF for 3 consecutive days (dose 10 μg per day) followed (on the fourth day and exactly 4 weeks after ameroid implantation) by transendocardial injections of freshly aspirated ABM into the ischemic zone using a transendocardial injection catheter (2.4 ml per animal injected at 12 sites) and 8 control animals without GM-CSF stimulation were injected with heparinized saline. At baseline and 4 weeks later, animals underwent rest and pacing echocardiogram to assess regional contractility (% myocardial thickening), and microsphere study to assess collateral-dependent perfusion at rest and during adenosine infusion. Four weeks after injection of ABM collateral flow (expressed as the ratio of ischemic/normal zone×100) improved in ABM-treated pigs but not in controls (ABM: 85±11 vs. 72±16 at rest, P=0.026; 83±18 vs. 64±19 during adenosine, P=0.06; Controls: 93±10 vs. 89±9 at rest, P=0.3 1; 73±17 vs. 75±8 during adenosine, P=0.74). Similarly, contractility increased in ABM-treated pigs but not in controls (ABM: 93±33 vs. 63±27 at rest, P=0.009; 84±36 vs. 51±20 during pacing, P=0.014, Controls: 72±45 vs. 66±43 at rest, P=0.65; 70±36 vs. 43±55 during pacing, P=0.18).

The results indicate that catheter-based transendocardial injection of ABM prestimulated in vivo by GM-CSF administered systemically for 3 days, can augment collateral perfusion and myocardial function in ischemic myocardium, findings suggesting that this approach may constitute a novel therapeutic strategy for achieving optimal therapeutic angiogenesis.

### Example 6

Treatment of a Human Patient

Bone marrow (˜5 ml) will be aspirated from the iliac crest at approximately 60 minutes prior to initiation of the cardiac procedure using preservative-free heparinized glass syringes (20 unit heparin/l ml fresh BM). The aspirated bone marrow will be immediately macro-filtered using 300μ and 200μ stainless steel filters, sequentially. An experienced hematologist will perform the procedure under sterile conditions. The bone marrow smear will be evaluated to confirm a normal histomorphology of the bone marrow preparation.

Any of several procedures for delivery of an agent to the myocardium can be used. These include direct transepicardial delivery, as could be achieved by a surgical approach (for example, but not limited to, a transthoracic incision or transthoracic insertion of a needle or other delivery device, or via thoracoscopy), or by any of several percutaneous procedures. Following is one example of percutaneous delivery. It should be emphasized that the following example is not meant to limit the options of delivery to the specific catheter-based platform system described in the example—any catheter-based platform system can be used.

Using standard procedures for percutaneous coronary angioplasty, an introducer sheath of at least SF is inserted in the right or left femoral artery. Following insertion of the arterial sheath, heparin is administered and supplemented as needed to maintain an ACT for 200-250 seconds throughout the LV mapping and ABM transplantation portion of the procedure. ACT will be checked during the procedure at intervals of no longer than 30 minutes, as well as at the end of the procedure to verify conformity with this requirement.

Left ventriculography is performed in standard RAO and/or LAO views to assist with guidance of NOGA-STAR3 and injection catheters, and an LV electro-mechanical map is obtained using the NOGA-STAR3 catheter. The 8F INJECTION-STAR catheter is placed in a retrograde fashion via the femoral sheath to the aortic valve. After full tip deflection, the rounded distal tip is gently prolapsed across the aortic valve and straightened appropriately once within the LV cavity.

The catheter (incorporating an electromagnetic tip sensor) is oriented to one of the treatment zones (e.g., anterior, lateral, inferior-posterior or other). Utilizing the safety features of the NOGA3 system, needle insertion and injection is allowed only when stability signals will demonstrate an LS value of <3. A single injection of 0.2 cc of freshly aspirated ABM will be delivered via trans-endocardial approach to the confines of up to two treatment zones with no closer than 5 mm between each injection site. The density of injection sites will depend upon the individual subject's LV endomyocardial anatomy and the ability to achieve a stable position on the endocardial surface without catheter displacement or premature ventricular contractions (PVCs).

That freshly aspirated ABM transplanted into ischemic myocardium is associated with improved collateral flow without adverse effects may be of clinical importance for several reasons. The methodology reflected above took advantage of the natural capability of the bone marrow to induce a localized angiogenic response in an effective and apparently safe manner. Such an angiogenic strategy would probably be less costly than many others currently being tested. It would also avoid potential toxicity-related issues that are remote but definite possibilities with various gene-based approaches using viral vectors.

The invention is based on the concept that ABM may be an optimal source for cellular (an example would be mononuclear stromal cells, but the invention is not limited to such cells as many other cells in the bone marrow may contribute importantly to the angiogenic effect) and secreted, e.g., angiogenic growth factors, elements necessary to promote new blood vessel growth and restore function when transferred to another tissue, such as ischemic heart or peripheral limbs. A patient's own bone marrow can be used as the key therapeutic source to induce therapeutic angiogenesis and/or myogenesis in ischemic tissues, e.g., heart muscle and/or ischemic limb, with compromised blood perfusion due to arterial obstructions. The patient's own bone marrow is aspirated, i.e., ABM donation, processed as described herein, and injected directly into ischemia and/or adjacent non-ischemic tissue, e.g., heart muscle and/or ischemic limb muscle, to promote blood vessel growth.

The ABM and/or bone marrow products are injected into the heart muscle, e.g., the myocardium, by use of either a catheter-based trans-endocardial injection approach or a surgical (open chest or via thoracoscopy) trans-epicardial thoracotomy approach. Those two delivery strategies can be used to achieve the same therapeutic goal by promoting the incorporation and integration of angiogenic bone marrow elements in the target organ tissue, e.g., heart muscle and/or ischemic limb.

According to this aspect of the invention, effective amounts of ABM, bone marrow cells or bone marrow cells transfected with an angiogenesis-promoting agent are administered for treatment. As would be appreciated by experienced practitioners, the amount administered will depend upon many factors, including, but not limited to, the intended treatment, the severity of a condition being treated, the size and extent of an area to be treated, etc. With regard to treatment according to the invention, a representative protocol would be to administer quantities of from about 0.2 to about 0.5 ml of ABM in each of from about 12 to about 25 injections, for a total of from about 2.4 to about 6 ml of ABM being administered. Each dose administered could preferably comprise from about 1 to about 2 percent by volume of heparin or another blood anticoagulant, such as coumadin. When the ABM has been cultured or stimulated and/or is being administered in combination with other pharmaceuticals or the like, the quantity of ABM present should be approximately the same in each dose and/or the total of the ABM administered should be about the same as described above. It is believed that the total number of cells of ABM administered in each treatment should be on the order of from about 10⁷ to 5×10⁸.

In another embodiment of the invention, optimization of angiogenic gene expression may be enhanced by co-administration of various angiogenic stimulants with the ABM. Thus, according to the invention ABM transplantation is injected either as a “stand alone” therapeutic agent or combined with any pharmacologic drug, protein or gene or any other compound or intervention that may enhance bone marrow production of angiogenic growth factors and/or promote endothelial cell proliferation, migration, and blood vessel tube formation. The “combined” agent(s) can be administered directly into the patient or target tissue, or incubated ex-vivo with bone marrow prior to injection of bone marrow into the patient. Examples of these “combined” agents (although not limited to these agents) are Granulocyte-Monocyte Colony Stimulatory Factor (GM-CSF), Monocyte Chemoattractant Protein 1 (MCP 1), EPAS1, or Hypoxia Inducible Factor-1 (HIF-1). The stimulation of the bone marrow could be by the direct exposure of the bone marrow to the factors in the form of proteins, or the bone marrow cells can be transfected with vectors carrying the relevant genes. For example, bone marrow can be transfected with a plasmid vector, or with an adenoviral vector, carrying the HIF-1 or EPAS1 transgenes. An example of an intervention that may enhance bone production of angiogenic factors is ex-vivo exposure of bone marrow cells to hypoxia. This intervention can be used alone with bone marrow, or in combination with any of the factors outlined above. These optimization strategies are designed to increase the production of vascular endothelial growth factor (VEGF) expression and/or other cytokines with angiogenic activity prior to the direct injection of the bone marrow into the heart or any peripheral ischemic tissue. In a broad sense, the invention comprises intramyocardial injection of ABM with any agent that would become available to cause stimulation of bone marrow and/or ex-vivo or in vivo stimulation of any angiogenic growth factor production by the bone marrow or its stromal microenvironment.

Delivery of the above-described therapeutic modalities to patients will vary, dependent upon the clinical situation. For example, patients with refractory coronary artery disease or ischemic peripheral vasculopathy are candidates for a bone marrow aspiration procedure followed by ABM myocardial or limb transplantation directed into the ischemic tissue or its borderline zone and/or normal tissue that may serve as the source for collateral or cellular supply to the diseased tissue for the purposes of therapeutic angiogenesis and/or myogenesis. For example, patients with refractory coronary artery disease or ischemic peripheral vasculopathy are candidates for a bone marrow aspiration procedure followed by ABM myocardial or limb transplantation directed into the ischemic tissue or its borderline zone and/or normal tissue that may serve as the source for collateral or cellular supply to the diseased tissue for the purposes of therapeutic angiogenesis and/or myogenesis. This procedure involves the use of a bone marrow aspiration procedure, bone marrow harvesting and processing, followed by the use of the ABM or its elements (growth factors and/or cellular elements being isolated from the patient's own bone marrow), with or without any ex-vivo stimulation of its delivery forms, to be injected into the ischemic or non ischemic myocardium and/or peripheral ischemic muscle tissue (such as limb muscle ischemia). The bone marrow will be kept in standard anticoagulation/anti-aggregation solution (containing sodium citrate and EDTA) and kept in 4° C. in sterile medium until the time of its use.

Upon its use, the bone marrow is filtered to avoid injecting remaining blood clots or macro aggregates into the target tissue.

The bone marrow, with or without a stimulatory agent in any of its delivery forms, or with or without having been transfected with a vector carrying a transgene that is designed to enhance the angiogenesis effect of the bone marrow, is injected into the heart muscle, i.e., in therapeutic myocardial angiogenesis or therapeutic myogenesis, using either any catheter-based trans-endocardial injection device or via a surgical (open chest) trans-epicardial thoracotomy approach, or any other approach that allows for transepicardial delivery. In the case of treatment of limb muscle ischemia the bone marrow cells (i.e., early attaching cells) are transferred by a direct injection of the bone marrow or it elements, with or without ex-vivo or in vivo stimulation in any of its delivery forms, into the muscles of the leg.

The volume of injection per treatment site can range between 0.1-5.0 cc per injection site, dependent upon the specific bone marrow product, severity of the ischemic condition and the site of injection. The total number of injections can range between 1-50.injection sites per treatment session.

### Example 7

Pig Bone Marrow Culture

Bone marrow cells (BMCs) were harvested under sterile conditions from pigs in preservative free heparin (20 units/ml BM cells) and filtered sequentially using 300μ and 200μ stainless steel mesh filters. BMCs were then isolated by Ficoll-Hypaque gradient centrifugation, seeded in T-75 flasks, and cultured overnight in long-term culture medium (LTCM) (Stem Cell Tech, Vancouver, British Columbia, Canada) at 33° C. with 5% CO₂ in T-75 culture flasks. The medium was then changed and the non-attaching cells washed out. The attached cells (i.e., “early attaching cells”) were mostly mononuclear cells, endothelial precursor cells, or other hemopoietic lineage cells. Among the mononuclear cells in early attaching cells are marrow-derived stromal cells. By lac-Z staining testing, these cells have been shown to be permissive for adenovirus by expression of the marker protein.

The seeding density of the BMCs in each culture dish is 7×10⁶/ml. When the cells become confluent at about 7 days, they are split 1 to 3 by 0.25% trypsin. Passages 3-8 were used for this study.

Adenovirus Transfection

BMCs were first cultured in 6-cm Petri dishes for 3 to 14 days to allow for production of a lining of early attaching cells that adhere to the Petri dish. The non-adherent cells are washed away the day after initial seeding. Then the early attaching cells were inoculated with a vector encoding one or more cytokines, growth factors, or other mammalian angiogenesis promoting factors, such as, but not limited to, the transcription factors HIP-1 or HIF-2. This inoculation can occur from 3 to 28 days after seeding, for example 3 to 12 days or 3 to 8 days. The virus was washed out from the transfected cells about 2 hours to 3 days after inoculation. The transfected cells were then injected into the patient's target tissue, such as the muscle of heart or leg.

### Example 8

MSCs have the Capacity to Secrete Biologically Active Collateral-Enhancing Factors in vitro.

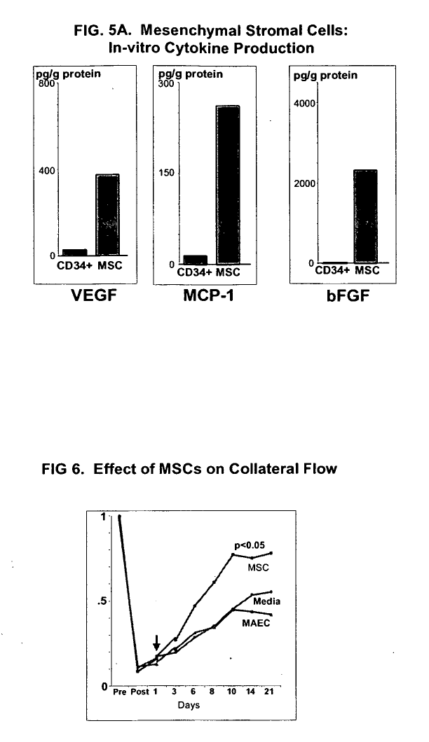

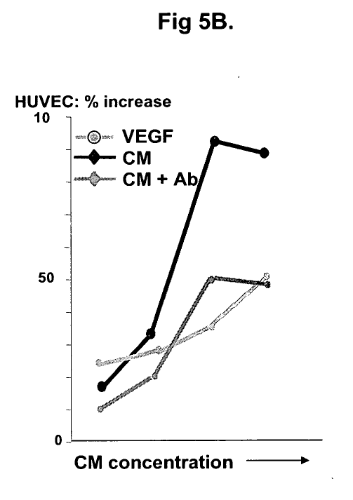

As a first test of the feasibility of the hypothesis that HIF-1 transduction of MSCs increases the angiogenic potential of the cells, murine MSCs were cultured and the conditioned medium was serially analyzed for cytokine production (FIG. 5). Mononuclear marrow cells were harvested from the femur and tibiae of mice and the mononuclear fraction separated using a Ficoll density gradient. The cells were cultured for 10 days and the CD34 minus/CD45 minus cells were isolated from the heterogeneous cultured cells using a double magnetic bead technique. This isolation procedure involves negatively selecting cells not expressing cell markers CD34 and CD45 by using magnetic beads labeled with commercially available antibodies to these markers MSCs were purified from the heterogeneous cultured cells. The CD34 minus-/CD45 minus-fraction was isolated by labeling with FITC-labeled anti-CD34 antibody (Pharmingen, San Diego, Calif.) followed by simultaneous incubation with anti-FITC and anti-CD45 magnetic beads (Miltenyi Biotech, Sunnyvale, Calif.). Cells were passed through a magnetic column and the double-negative fraction collected. Subsequently, the bead-negative and bead-positive populations were separately cultured. The bead-negative population demonstrated typical fibroblastic morphology of the MSCs, while the bead-positive population appeared to mainly consist of small, spherical cells consistent with lymphohematopoietic cells (FIGS. 5A and 5B). FACS analysis was performed and demonstrated that cells did not express the surface makers CD3 1, CD34, CD45, and CD 117 typical of lymphohematopoietic cells, but did express high levels of CD44 (95±0.6%), CD90 (99.1±0.1%), and CD105 (89±2.1%) typical of marrow derived-stromal cells. (These CD34 minus/CD45 minus cells are also referred to herein as “marrow-derived stromal cells”, or “MSCs”). The isolated MSCs were replated, and the conditioned media subsequently collected for 24 hours.

Conditioned media prepared as above was analyzed for the presence of angiogenic cytokines by ELISA. Cytokine levels were corrected for total cell culture protein. The data reflect at least 3 different cell populations, with each population containing cells pooled from 2 mice. The results show (FIG. 5) that MSCs express such known collateral-enhancing factors as VEGF, MCP-1, and bFGF (also, angiopoietin-1 and P1DGF (not shown)). In contrast, CD34+ cells (progenitor endothelial cells) do not express these factors.

The functional capacity of the cytokines secreted into the medium of cultured MSCs was also tested by testing their capacity to cause endothelial cell proliferation. MSC-conditioned media prepared as above was collected and found to indeed increase the proliferation of cultured human umbilical vein endothelial cells. MAECs or SMC's (1×10⁴/well) were plated in 24-well plates in MEM with 0.1% fetal calf serum for 24-hours. The media was then replaced with varying dilutions of MSC^{CM} or control wells of DM-10 only. Cultures were continued for 72-hours, after which the cells were recovered and counted using a Coulter counter. Data is reported as the mean % change in proliferation when compared with control.

To study the effect of VEGF alone for comparison, endothelial cells were cultured in non-conditioned medium containing recombinant VEGF protein in concentrations found in conditioned media (using the above ELISA data). To block the effect of VEGF in the conditioned media, 10 μg of VEGF blocking antibody was added. The results shown in FIG. 5B show that bone marrow-derived progenitor cells, such as MSCs secrete factors that increase endothelial cell proliferation. VEGF is one of the factors that contribute to this effect as is shown by blocking antibody to VEGF reducing cell proliferation compared to conditioned medium alone. The “VEGF curve” in FIG. 5B was derived from conditions in which recombinant VEGF was added to non-conditioned medium (not exposed to MSCs) in a concentration identical to that measured in the MSC-conditioned medium, resulting in endothelial cell proliferation considerably less than that achieved with conditioned medium. This result indicates that VEGF is only one of the factors contributing to the proliferative effects of media conditioned by MSCs. Studies also demonstrated that MSC conditioned media increased migration of endothelial cells.

MSCs Increase Collateral Flow in the Mouse Ischemic Hind Limb.

Twelve week-old Balb/C male mice underwent right distal femoral artery ligation using a method known in the art. Twenty-four hours later, mice were randomized to 3 groups—one group received 1×10⁶ MSCs prepared as above described from syngenic mice, one group received 1×10⁶ mature endothelial cells isolated from syngenic mice, and one group received non-conditioned media injected into the adductor muscles of the ischemic hind limb. Laser Doppler perfusion imaging (LDPI) was utilized to follow ischemic hind limb flow recovery over the ensuing 28 days (FIG. 6).

The results of these tests shown in FIG. 6 demonstrate that injection of MSCs into the adductor muscles of the ischemic hind limb significantly increased collateral flow, an effect not seen by injecting mature endothelial cells.

MSCs Injected into the Region of Developing Collaterals Express Collateral-Enhancing-Related Cytokines in vivo and Incorporate into Developing Collaterals.

MSCs were incubated ex-vivo with carboxyfluorescein diacetate succinimidyl esters (CFSD) to stain cytoplasm green and injected into adductor tissue of the ischemic hind limb of mice as described. Mice were dilled a t 7 days and adductor muscles snap frozen. Eight-micron sections were taken and stained for VEGF using HRP-conjugated anti-VEGF antibodies. Micrographs of the stained sections showed CFSE positive MSCs surrounded by VEGF staining, reflecting secreted VEGF. Thus, the injected MSCs express VEGF in vivo after injection. Western blotting of the adductor muscle confirmed the increase in VEGF levels caused by MSC injection and also showed increased levels of bFGF. These results show that in an ischemic animal model, the increase in collaterals induced by MSC injection is caused, at least in part, by the capacity of the MSCs to express and secrete VEGF and bFGF.

Confirmation of Cellular Survival and Gene Product Expression Following Transduction of MSCs.

As an initial step to determine whether MSCs provide an appropriate target for genetic alteration, the viability of MSCs in-situ following ex-vivo transduction with an adenoviral vector was examined. To this purpose, two separate experiments were performed, one utilizing an adenovirus comprising a gene encoding for Green Fluorescent Protein (GFP) and one comprising a gene encoding β-galactosidase. MSCs prepared as above were transduced ex-vivo. Preliminary studies determined that over 90% of MSCs were successfully transduced with an adenovirus containing a reporter transgene at an MOI of 150 (data not shown). To track protein expression, cells were incubated with Ad.GFP or Ad.β-galactosidase at an MOI of 150 for 2-hours, rinsed three times, recovered and immediately injected into the adductor muscle (24-hours post-surgery). To follow the fate of injected GFP+/MSCs, multiple sections of adductor and calf muscle were examined using a Nikon inverted fluorescent microscope. To follow the fate of β-gal+/MSCs, sections were developed with a commercially available X-gal kit (Invitrogen) and immediately injected into the adductor muscle of mice that had undergone femoral artery ligation 24-hours previously. Mice were sacrificed at day-3, day-7 and day-l 4. Adductor muscle sections were subsequently either examined under a fluorescent microscope or stained with X-gal depending on the appropriate protocol as known in the art.

At day-3, few cells were found that expressed the gene-of-interest. However, by day-7 and maintained through to day-14, many cells expressing the gene-of-interest were found distributed throughout the adductor tissue.

Therefore, this experiment not only confirmed cell viability and preservation of the transcriptional/translational mechanism, but also demonstrated that MSCs can be used as a vector to introduce genes-of-interest into a particular tissue, such as muscle tissue.

HIF-1α/VP16 transfection of MSCs in vitro Leads to an Increase in Collateral-Enhancing-Related Factors Greater than Those Induced by Hypoxia.

Murine MSCs were isolated and plated as described above. Three groups of MSCs were compared. Group 1—MSCs cultured under normoxic conditions; Group 2—MSCs cultured in 1% O₂; Group 3—MSCs transfected with an adenovirus encoding HIF-1α/VP16 prepared as described above. MSCs were incubated with the virus at a multiplicity of infection of 200 for 2 hr, followed by 48 hr of culture to allow time for gene expression.

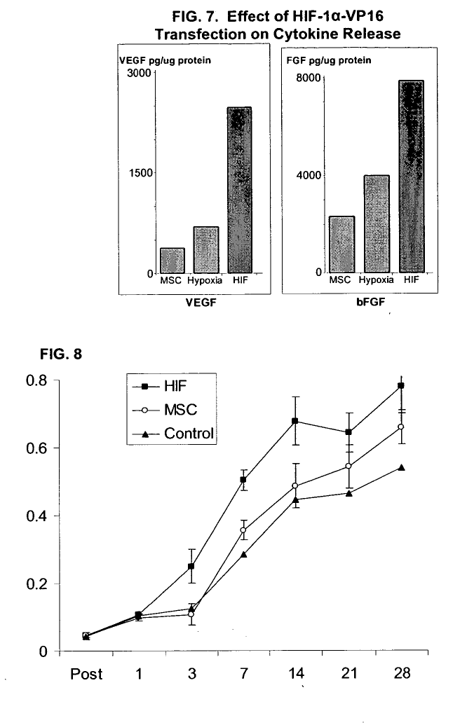

The culture-conditioned media was subsequently collected for 24 hr from all 3 groups of cells. Using commercially available ELISA kits, media was analyzed for the presence of angiogenic cytokines VEGF and b-FGF. Cytokine levels were corrected for total cell culture protein. The results shown in FIG. 7 demonstrate that HIF-1α/VP16 transfection increases expression and secretion by MSCs of both VEGF and b-FGF to levels substantially greater than those achieved by hypoxia.

Conditioned medium bathing these cultured cells (MSC conditioned medium, or MSC^{CM}) was also added to cultures of endothelial cells (EC) and smooth muscle cells (SMC) to assess the effect of MSC^{CM} on cell proliferation. Mouse aortic endothelial cells (MAECs) were isolated as follows. Under sterile conditions, murine thoracic aortas were dissected (n=10), the adventitia removed, and then cut into 1-2 mm rings. Rings were then incubated with 0.25% trypsin for 20 minutes at 37° C., followed by washing and harvesting of floating cells. These were cultured in Minimal Essential Media supplemented with 10% FBS. Cells were uniformly positive for Factor VIII. Smooth muscle cells (SMCs) were isolated using a modification of a previously described protocol.⁸ Briefly, after collecting MAECs as above, collagenase in Hanks Balanced Salt Solution (1 mg/ml) was added and incubated in 37° C. for up to 3 hours with gentle agitation every 15-30 min. Floating cells were again harvested, washed and re-suspended in Medium 199 supplemented with 10% FBS. Cells stained uniformly for smooth-muscle actin. Passages 3-8 for both cells were used for the purposes of the study.

When compared to MSC^{CM} from MSCs under conditions of normoxia or hypoxia, MSC^{CM} from HIF-1α/VP1 6-transduced MSCs increased EC proliferation (290% vs. 31% vs. 79% compared to proliferation in control media, p<0.001) and SMC proliferation (220% vs. 26% vs. 58%, p<0.001).

Conditioned media from non-transfected MSCs and MSCs transfected with HIF-1α/VP16 vector were collected as described above. Human umbilical vein endothelial cells were cultured in varying dilutions of the conditioned media for 72 hr. Cells were then trypsinized and counted. Conditioned medium derived from the MSCs transfected with HIP-1α/VP16 vector markedly increased HUVEC proliferation. The increase was even greater than that observed following the addition to endothelial cells of conditioned medium from non-transfected cells subjected to 24 hr of hypoxia. These results demonstrate that the increase in MSC cytokine production following transfection with HIP-1α/VP16 vector produces significant in vitro cytokine production from MSCs of the type that increases collateral development in tissue into which these cells are implanted.

HIF-1α/VP16 Transfection of MSCs Leads to an Increase in Collateral Flow.

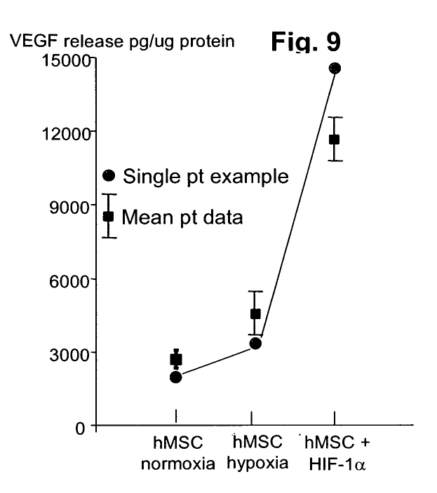

The effects of HIF-1α/VP16 transduction of MSCs on collateral flow in a mouse model of hind limb ischemia was studied next. One group of animals (as above) received 1×10⁵ non-transduced MSCs, one group received HIF-1α/VP16-transduced cells and a third group received media. Flow in the ischemic limb was monitored as described above. The results collected over the course of 21 days (FIG. 8) showed that mice treated with transduced MSCs demonstrated a consistently greater increase in collateral flow recovery than that observed in mice treated with non-transduced MSC.

In summary, this experiment shows that transfection with HIF-1α/VP16 in an adenoviral vector significantly and markedly enhances the in-vitro angiogenic effects of MSCs. More importantly, in vivo studies indicate that this strategy results in an increase in the collateral-improving effects over that achieved by injection of MSCs alone. These studies indicate that transduction of MSCs with HIF-1α, (and also with genes encoding other angiogenic-related cytokines, such as the FGF family of proteins, and NOS) will optimize the collateral-enhancing effects of a cell-based strategy for increasing collateral flow in ischemic tissue.

### Example 9

Depressed Levels of VEGF Secretion Observed in the MSCs of Coronary Artery Disease Patient are Substantially Increased by Ad.HIF-1α/VP16 Transduction.

This example demonstrates that production of angiogenic cytokines by bone marrow-derived progenitor cells can be substantially increased by transduction with a viral vector encoding recombinant HIF-1α/VP16. Freshly aspirated and filtered BM cells of human coronary artery disease were used to obtain hMSCs as described above. The hMSCs were transfected with vector HIF-1α/VP16 and cultured to obtain conditioned medium as above. Levels of VEGF in the conditioned medium of transfected hMSCs were measured by ELISA and compared with those of untransfected hMSCs under normoxic and hypoxic conditions (FIG. 9). Data of the patient with lowest VEGF expression at baseline is shown separately. The results of this experiment demonstrated that human MSCs obtained from coronary artery disease patients with very low levels of VEGF expression can be converted by transduction with Ad.HIF-1α/VP16 to higher than average levels of VEGF expression.

The present invention may be embodied in other specific forms without departing from the spirit or central attributes thereof. Thus, the foregoing description of the present invention discloses only exemplary embodiments thereof, and other variations are contemplated as being within the scope of the present invention. Accordingly, the present invention is not limited to the particular embodiments that have been described in detail herein. Rather, reference should be made to the appended claims as indicative of the scope and content of the invention.

The preceding specific embodiments are illustrative of the practice of the invention. It is to be understood, however, that other expedients known to those skilled in the art or disclosed herein, may be employed without departing from the spirit of the invention or the scope of the appended claims.

What is claimed is:

1. A method for promoting collateral blood vessel formation and tissue perfusion in tissue, said method comprising:
a) injecting conditioned medium of bone marrow early attaching cells into the tissue to promote formation of collateral blood vessels in the tissue.

2. The method of claim 1, wherein the early attaching cells are marrow-derived stromal cells.

3. The method of claim 1, further comprising prior to a) transfecting at least a portion of the bone marrow early attaching cells with a vector comprising a polynucleotide that encodes a fibroblast growth factor (FGF) and/or a nitric oxide synthase (NOS), wherein the conditioned medium comprises the FGF and/or the NOS expressed by the vector in the cells.

4. The method of claim 3, wherein the polynucleotide encodes a FGF.

5. The method of claim 4, wherein the FGF is selected from FGF-1, FGF-2, FGF-4, and FGF-5.

6. The method of claim 3, wherein the polynucleotide encodes a NOS.

7. The method of claim 6, wherein the NOS is selected from NOS is selected from inducible NOS and endothelial NOS.

8. The method of claim 3, wherein the vector is selected from a plasmid vector and an adenoviral vector.

9. The method of claim 8, wherein the vector is an adenoviral vector.

10. The method of claim 8, wherein transfection is by co-culture of the adenoviral vector with the early attaching cells.

11. The method of claim 1, wherein the bone marrow early attaching cells are autologous.

12. The method of claim 1, wherein the bone marrow early attaching cells are not autologous.

13. The method of claim 1, wherein bone marrow cells are grown in vitro for about 12 hours to about 12 days prior to isolation of the bone marrow early attaching cells.

14. The method of claim 3, further comprising stimulating the transfected early attaching cells with hypoxia.

15. The method of claim 1, wherein the tissue is muscle tissue.

16. The method of claim 1, wherein the conditioned medium is injected into the muscle tissue.

17. The method of claim 15, wherein the conditioned medium is injected into muscle tissue adjacent to ischemic muscle tissue.

18. The method of claim 1, wherein the cells are injected directly into heart or leg muscle to promote angiogenesis therein.

19. The method of claim 18, wherein the method enhances collateral blood vessel formation in the heart or leg muscle.

20. A composition comprising conditioned medium of bone marrow early attaching cells.

21. The composition of claim 20, wherein the early attaching cells are bone marrow derived stromal cells.

22. The composition of claim 20 further comprising a transgenically expressed one or more angiogenic agent selected from VEGF, FGF, NOS, or a combination thereof.

23. The composition of claim 22, wherein the FGF is selected from FGF-1, FGF-2, FGF-4, and FGF-5.

24. The composition of claim 22, wherein the NOS is selected from inducible NOS and endothelial NOS.

25. The composition of claim 22, further comprising a vector expressing the angiogenic agent.

26. The composition claim 25, wherein vector is an adenoviral vector.

27. The composition of claim 22, wherein the transfected cells are stimulated by exposure to hypoxia.

28. The composition of claim 20, further comprising an anticoagulant.

29. The composition of claim 26, wherein the anticoagulant is heparin or coumadin.
